SEQUENCE LISTING (13 sequences)

SEQ ID NO: 1 - DNA, 20 bp, Homo sapiens
  misc_feature: Primer sequence
accccaagagtggagttgtg

SEQ ID NO: 2 - DNA, 20 bp, Homo sapiens
  misc_feature: Primer Sequence
ggaaggcattttctgatcca

SEQ ID NO: 3 - DNA, 20 bp, Homo sapiens
  misc_feature: Primer Sequence
caggtttggagtgggacagt

SEQ ID NO: 4 - DNA, 20 bp, Homo sapiens
  misc_feature: Primer Sequence
ctccattgcacactggtgac

SEQ ID NO: 5 - DNA, 20 bp, Homo sapiens
  misc_feature: Primer Sequence
tctgattcccaagagggttg

SEQ ID NO: 6 - DNA, 20 bp, Homo sapiens
  misc_feature: Primer Sequence
ctgcctgatcaagagcatca

SEQ ID NO: 7 - DNA, 20 bp, Homo sapiens
  misc_feature: Primer Sequence
gaacaggccaagactgaagc

SEQ ID NO: 8 - DNA, 20 bp, Homo sapiens
  misc_feature: Primer Sequence
gcccctggttagaatcttcc

SEQ ID NO: 9 - DNA, 20 bp, Homo sapiens
  misc_feature: Primer Sequence
gacaccactggagggtgact

SEQ ID NO: 10 - DNA, 20 bp, Homo sapiens
  misc_feature: Primer Sequence
caggtccacatggtcttcct

SEQ ID NO: 11 - DNA, 20 bp, Homo sapiens
  misc_feature: Primer Sequence
ttttcagagctttgggcact

SEQ ID NO: 12 - DNA, 20 bp, Homo sapiens
  misc_feature: Primer Sequence
ccattttgccagaaattcgt

SEQ ID NO: 13 - RNA, 21 nt, Homo sapiens
  misc_feature: Artificial Sequence
aagaaucgagagcuccagauc

(19) United States
(12) Patent Application Publication
(10) Pub. No.: US 2007/0196514 A1
(43) Pub. Date: Aug. 23, 2007

(54) Prostate cancer treatment with glycogen synthase kinase-3beta inhibitors

(76) Inventor: Benyi Li, Overland Park, KS (US)

Correspondence Address: STINSON MORRISON HECKER LLP;ATTN: PATENT GROUP, 1201 WALNUT STREET, SUITE 2800, KANSAS CITY, MO, 64106-2150, US

(21) Appl. No.: 11/358,275

(22) Filed: Feb. 21, 2006

(51) Int. Cl. A61K 48/00; A61K 33/00; A61K 31/616; A61K 31/4245; A61K 31/433; A61K 31/404

(52) U.S. Cl. 424/722; 514/165; 514/44; 514/557; 514/414; 514/341; 514/362; 514/364; 514/215

(57) ABSTRACT

A method of treating prostate cancers, including androgen refractory prostate cancers, using glycogen synthase kinase-3β inhibitors, such as lithium.

### CROSS-REFERENCE TO RELATED APPLICATIONS

Not Applicable.

### STATEMENT REGARDING FEDERALLY SPONSORED RESEARCH OR DEVELOPMENT

Not applicable.

### FIELD OF THE INVENTION

The present invention generally relates to compositions and the treatment and prevention of prostate cancer. In particular, the present invention is directed to the use of glycogen synthase kinase-3β (“GSK-3β”) inhibitors, such as lithium, to treat prostate cancer by a mechanism involving S-phase arrest of the cell cycle.

### DESCRIPTION OF RELATED ART

Glycogen synthase kinase 3 (“GSK-3”) belongs to a family of conserved serine/threonine kinases present in all eukaryotic groups. There are two isoforms in humans, namely, GSK-3α (51 kDa), and GSK-3β (47 kDa). Recent studies have shown that GSK-3 is involved in many biological processes, including cell cycle progression, gene transcription, apoptosis and survival, cellular metabolism, cell movement, tumorigenesis, cytokinesis, as well as embryonic development (Jope 2004). Regulation of GSK-3 activity is not a simple linear event but a dynamic and rather complicated process, depending on the upstream stimuli and the contact surrounding the enzyme inside a cell. These processes include activating and inhibiting mechanisms, scaffolding complexes, and differential recognition of target proteins (Kim 2000). Usually, phosphorylation of GSK-3β on serine 9 will inactivate the enzyme whereas phosphorylation on tyrosine 216 enhances its enzymatic activity. One of its functions as protein kinase, GSK-3 phosphorylates many transcription factors to regulate their activities, such as C/EBP (Ross 1999).

Previous studies have directly linked GSK-3 over expression or activation with human cancer development and progression (reviewed in Yang 2004), although no mutation on GSK-3 gene was found in cancer patients (Manoukian 2002). Recently, the role of GSK-3β in certain prostate cancer cell lines has been investigated in vitro in two distinct pathways. The first pathway involves the effect of GSK-3β in androgen receptor (“AR”) transactivation in androgen-responsive prostate cancer cells. It was shown that GSK-3β activity was required for androgen receptor transactivation in vitro (Liao 2004). Another research group showed that GSK-3β inhibitors reduced the growth of prostate cancer cell in vitro in AR-expressing prostate cancer cell lines (Mazor 2004). That same group found that the GSK-3β inhibitor SB216763 did not affect growth in AR-null PC-3 cells and concluded that the AR was required for the growth inhibitory response. Controversy still exists about the role of GSK-3β in cancer progression involving the AR-pathway (Wang 2004, Salas 2004).

The second pathway involving GSK-3β in prostate cancer involves activation of an apoptotic pathway. It was shown that the inhibition of GSK-3β activity in vitro sensitized prostate cancer cells to tumor necrosis factor (“TNF”) related apoptosis-inducing ligand “TRAIL”)-mediated apoptosis when exogenous TRAIL was added to cell cultures (Liao 2003). Thus, searchers have reported that there appears to be a requirement for GSK-3β activity in TNF regulation of cell survival (Hoeflich 2000).

Lithium is a GSK-3β inhibitor. Lithium-induced attenuation of cell proliferation in normal or neoplastic cells has been noted for a long time (Huot 1972). In a cell culture system, lithium inhibited cancer cell growth derived from hepatocellular carcinoma (Erdal 2005). The possible preventive nature of lithium in human cancer development was recently reported (Cohen 1998). In that study, the risk of cancer development generally among lithium-treated patients with mental disorders was reportedly significantly lower than that in the untreated group or in the general population. Additionally, a significant inverse relationship was shown between cancer development and lithium dose.

The present invention is directed to the inhibition of prostate cancer via a surprising new pathway involving S-phase arrest in androgen refractory cells, as opposed to pathways involving AR transactivation inhibition or TRAIL-mediated apoptosis. In addition, in vivo studies surprisingly showed that a therapeutically effective dose of the GSK-3β inhibitor, LiCl, was significantly lower than that conventionally used to treat mood disorders.

### BRIEF SUMMARY OF THE INVENTION

The present invention is directed to a method of treating prostate cancer, and in particular to androgen-refractory prostate cancers, using GSK-3β inhibitors.

In one aspect, the present invention is directed to the anti-tumor effects of GSK-3β inhibitors in treating and preventing prostate cancer. In an in vitro cell culture model, it was found that GSK-3β inhibitors suppress cancer cell growth, induce S-phase cell cycle arrest, and abolish DNA replication in a time-dependent and dose-dependent manner. Moreover, the suppressive effects of GSK-3β inhibitors on prostate cancer cells were determined to be associated with down-regulation of DNA replication-related genes including cdc6, cyclin A and E, as well as cdc25C. In parallel, GSK-3β inhibitors up-regulate CDK inhibitor p21^{CIP1}. In addition, GSK-3β inhibitors were found to significantly suppress tumor growth in a mouse xenograft model without any appreciable side effects.

In another aspect of the present invention, it was shown that GSK-30 inhibitors are effective in treating prostate cancer in a TRAIL-independent mechanism that does not involve the administration of exogenous TRAIL.

In still another aspect of the present invention, the GSK-3β inhibitors are shown to inhibit prostate cancer by arresting the cell cycle in S-phase. Once the cell is arrested, the cell will not grow again, and tumor growth terminates.

In a further aspect, the GSK-3β inhibitors of the present invention are expected to inhibit pain associated with metastasis of prostate cancer to the bone tissue of the patient. Bone metastasis is one of the most frequent complaints of advance prostate cancer patients. By arresting cell growth, the tumor growth will be inhibited, which will reduce the pain for bone metastatic cancer patients.

In still another aspect, it is anticipated that the prostate cancer cells arrested in S-phase will be more sensitive to other cytotoxic drugs, such as Taxol (paxitaxel), and radiotherapy. See Bortul, et al., Deguelin, A PI3K/AKT inhibitor, enhances chemo sensitivity of leukemia cells with an active PI3K/AKT pathway, Br J Haematol. 129(5):677-86 (June 2005); Dong et al., E2F-1 overexpression sensitizes colorectal cancer cells to camptothecin, Cancer Gene Ther. 10(3):168-78 (March 2003). Kumi-Diaka, Chemosensitivity of human prostate cancer cells PC3 and LNCaP to genistein isoflavone and beta-lapachone, Biol Cell. 94(1):37-44 (Feb. 2002); O'Connor et al., Relationship between DNA cross-links, cell cycle, and apoptosis in Burkitt's lymphoma cell lines differing in sensitivity to nitrogen mustard, Cancer Res. 15; 51 (24):6550-7 (December 1991); Engelholm et al., Effect of melphalan on growth curves and cell cycle distribution of four human small cell carcinomas of the lung grown in nude mice, Exp Cell Biol. 54(3):138-48 (1986).

Additional aspects of the invention, together with the advantages and novel features appurtenant thereto, will be set forth in part in the description that follows, and in part will become apparent to those skilled in the art upon examination of the following, or may be learned from the practice of the invention. The objects and advantages of the invention may be realized and attained by means of the instrumentalities and combinations particularly pointed out in the appended claims.

### BRIEF DESCRIPTION OF THE DRAWINGS

FIG. 1 illustrates that LiCl inhibits cancer cell growth in vitro. In FIGS. 1A&B, AR-positive, androgen-dependent LNCaP cells and AR-null PC-3 cells were seeded in 12-well plates overnight and then treated with LiCl at indicated doses. Cells were harvested at indicated time-points, stained in 0.4% Trypan blue solution and then, the viable (white) cells were counted. In FIG. 1C, PC-3 cells were seeded in 35-mm dishes at a density of 1.0×10³ cells per dish with the culture medium containing LiCl at the indicated doses. The clonogenic survival fraction of the cells was determined at 14 days after platting. Colonies were fixed, stained, and photographed as described. Data represents three different experiments. The asterisk indicates a significant difference between LiCl versus the control.

FIG. 2 shows that GSK-3β inhibitors suppress cell growth as measured by 3-[4,5-dimethylthiazol-2-yl]-2,5-diphenyl tetrazolium bromide (“MTT”) assays. Androgen-dependent LNCaP cells (panel A), androgen-refractory C4-2 cells (panel B), and AR-null PC-3 cells (panel C) were seeded in 96-well plates and then treated with various reagents (GSK-3β inhibitors, NaCl or KCl) at different concentrations as indicated. Cell growth rate was determined three days later using a MTT assay kit as described herein. The value of optical density from controls was set as 100%, and then the value from treated cells was presented as a relative percentage value against the control. Data represents two separate experiments. The asterisk indicates a significant difference between LiCl versus control (panel A-C) or at the indicated dose levels of SB21673 and BIO versus untreated control (panel D).

FIGS. 3A&B shows that GSK-3β inhibitors induce S-phase arrest in LNCaP prostate cancer cells. Cells were serum starved for 24 hours and then returned to full culture media in the presence or absence of the inhibitors as indicated. Cells then were harvested at each time point and subjected to cell cycle analysis as described herein. The asterisk indicates a significant difference between LiCl versus control (panel B). FIGS. 3C&D shows the data for PC-3 and C4-2 cells. Data represents three different experiments.

FIG. 4 shows that GSK-3β inhibitors suppress DNA synthesis in S-phase. Cells were serum-starved for 24 hours and then left untreated or treated with the inhibitors as indicated in full culture media. DNA synthesis was determined using an enzyme-linked immunosorbent assay (“ELISA”)-based 5-bromo-2′-deoxyuridine (“BrdU”) incorporation assay as described herein. For panel C, the value of optical density from the control was set as 100%. The asterisk indicates a significant difference between indicated time-points (panel A) or drug-doses (panel B) versus control. Data was from two separate experiments.

FIG. 5 shows that GSK-3β inhibitors modulate gene expression. In FIG. 5A, androgen-dependent LNCaP cells were serum-starved for 24 hours and then treated with the inhibitors as indicated or the vehicle as control (lane 1). Total cellular RNAs were extracted and subjected for reverse transcription (“RT”) polymerase chain reaction (“PCR”) to determine the expression changes of genes as indicated. FIGS. 5B and 5C show the Western blot of LNCaP cells after serum starvation followed by treatment with vehicle, LiCl or BIO in full culture media and then harvested at different time point as indicated. Western blot was performed as described herein, and primary antibodies used were listed on the left side. Actin blot served as loading control. Data represents two different experiments.

FIG. 6 shows that GSK-3β silencing leads to altered gene expression and reduced cell growth. AR-null PC-3 cells were transfected with GSK-3β small-interfering RNA (“siRNA”) or a negative siRNA as described herein. A mock transfection was included as control. Cells were monitored for three days when the cell reached full confluence. Before cells were harvested for Western blot, a phase contrast image was taken from each well (panel B). Western blot was conducted as described herein, and primary antibodies used for Western blot were listed on the left side (panel A).

FIG. 7 shows that GSK-3β inhibitors suppress xenograft tumor growth. In FIG. 7A, AR-null PC-3 cells were inoculated subcutaneously into rear flanks of nude mice. The next day, animals (5 per group) were intraperitoneally (i.p.) injected with PBS (vehicle control) or LiCl (about 2 mg/kg body weight) daily. Xenograft tumor development and growth was monitored for eight weeks. FIG. 7B shows the average tumor weight for each group was determined after sacrificing the animals. FIG. 7C shows the tumor weight after PC-3 xenografts were palpable in nude mice and the animals (5 per group) were treated with PBS or LiCl for 2 weeks. An in vivo BrdU labeling assay was performed on those animals 2 hours before the end point of the treatment as described herein. The average tumor weight at the end of treatment was determined in each group. FIG. 7D shows that BrdU-labeled cells and apoptotic events in the xenografts were detected on paraffin-embedded tumor specimens as described herein, and H&E staining was carried out using standard technique. FIG. 7E shows the results of LNaP-BclxL xenografts (5 animals per group) in animals treated with SB216763 or vehicle control as described herein. Xenograft tumor growth was monitored and the average of tumor volume from each group was shown. The asterisk indicates a significant difference between treatments versus the control.

### DETAILED DESCRIPTION OF PREFERRED EMBODIMENT

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention. All publications and other references mentioned herein are incorporated by reference in order to describe the materials, methods, and examples discussed herein. In case of conflict, the present specification, including definitions, will control. The materials, methods, and examples are illustrative only and not intended to be limiting.

As use herein, prostate cells are “androgen responsive” or “androgen-dependent” if their growth is stimulated by androgens or hormones produced by the sex organs and adrenal glands.

As used herein, “androgen refractory” or “androgen insensitive” or “androgen independent” refers to the condition wherein normally androgen-dependent prostate cells become prostate tumor cells that do not depend on androgen for their proliferation.

As used herein, the “subject” or “patient” to be treated in the case of prostate cancer is preferably a mammal, and even more preferably a human.

As used herein, the term “treatment” as used herein with respect to prostate cancer refers to the treatment of the prostate cancer in a patient, such as a mammal (particularly a human), and includes: (a) preventing the prostate cancer from occurring, i.e., prophylactic treatment of a patient; (b) ameliorating the prostate cancer, i.e., eliminating or causing regression of the prostate cancer in a patient; (c) suppressing the cancer, i.e., inhibiting, slowing or arresting the development of cancer in a patient; or (d) alleviating the symptoms of the prostate cancer condition in a patient (e.g. pain caused by bone metastasis).

As used herein the term “inhibit” or “inhibiting” refers to a statistically significant and measurable reduction in activity, preferably a reduction of at least about 10% versus control, more preferably a reduction of about 50% or more, still more preferably a reduction of about 80% or more.

The present invention is directed to a method of treating prostate cancer, and in particular to treating androgen-refractory prostate cancers, by administering a therapeutically effective amount of a GSK-3β inhibitor to a subject or patient in need thereof.

The present invention provides for a pharmaceutical composition, which comprises a therapeutically effective amount of one or more GSK-3β inhibitors of the present invention or a pharmaceutically acceptable salt, ester or prodrug thereof, together with a pharmaceutically acceptable diluent or carrier.

Due to the high therapeutic potential of targeting GSK-3 in many different human diseases, so far more than thirty GSK-3 inhibitors have been identified or synthesized. Some of them have been co-crystallized with GSK-3β within the ATP-binding site of the protein (reviewed in Cohen 2004). Because of the close relationship between GSK-3β and CDKs, a given GSK-3β inhibitor often inhibits CDK activity. By contrast, as a non-ATP competitive GSK-3β inhibitor, lithium does not inhibit CDKs (Pheil 2001). In the present invention, GSK-3β inhibitors that do not inhibit CDKs are most preferred.

Suitable GSK-3β inhibitors include, but are not limited to lithium, GF109203X (2-[1-(3-Dimethylaminopropyl)-1H-indol-3-yl]-3-(1H-indol-3-yl)maleimide), RO318220 (2-[1-(3-(Amidinothio)propyl)-1H-indol-3-yl]-3-(1-methylindol-3-yl)maleimide methanesulfonate); SB216763 (3-(2,4-Dichlorophenyl)-4-(1-methyl-1H-indol-3yl)-1H-pyrrole-2,5-dione) (Santa Cruz Biotech, Santa Cruz, Calif.); SB415286 (3-[(3-Chloro-4-hydroxyphenyl)amino]-4-(2-nitrophenyl)-1H-pyrrole-2,5-dione) (GlaxoSmithKline, London, United Kingdom); 4-Benzyl-2-methyl-1,2,4-thiadiazolidine-3,5-dione (“TDZD-8”) (Axxora, San Diego, Calif.); 2-Thio(3-iodobenzyl-5-(1-pyridyl)-[1,3,4]-oxadiazole (“TIBPO”) (Axxora, San Diego, Calif.); 2,4-Dibenzyl-5-oxothiadiazolidine-3-thione (“OTDZT”) (Axxora, San Diego, Calif.); and 4-(2-Amino-4-oxo-2-imidazolin-5-ylidene)-2-bromo-4,5,6,7-tetrahydropyrrolo[2,3-c]azepin-8-one (“10Z-Hymenialdisine”) (Axxora, San Diego, Calif.). In addition, a number of monoclonal antibodies directed to GSK-3β are commercially available from Axxora. Other pharmacological inhibitors of GSK-3β are set forth in Meijer et al., “Pharmacological Inhibitors of Glyocogen Synthase Kinase 3, Trends in Pharmacological Sciences, Vol. 25, No. 9 (Sept 2004), which is incorporated by reference in its entirety.

The GSK-3β inhibitors may be administered to the patient in need in any form that will effectively deliver the agent. In the context of lithium, organic and organic salts are generally preferred. Suitable examples of pharmacologically acceptable salts, such as organic and inorganic salts, include lithium succinate, lithium citrate, lithium acetylsalicylate, lithium chloride, lithium carbonate, and lithium orotate, lithium succinate being generally preferred. Lithium may be administered in any suitable manner, including but not limited to orally, enterically, intravenously, peritoneally, or by injection. Commercially available lithium formulations include ESKALITH, ESKALITH CR, and LITHOBID.

The GSK-3β inhibitors may be administered in free form or in pharmaceutically acceptable salt, ester or prodrug form. Such salts, esters, and prodrugs may be prepared in conventional manner. As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio.

The compositions, including the GSK-3β inhibitors, may be formulated in any conventional form, for example, as tablets, capsules, caplets, solutions, suspensions, dispersions, syrups, sprays, gels, suppositories, patches, and emulsions.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically acceptable material, composition, or vehicle, such as a liquid or solid filler, diluent, excipient, solvent or encapsulating material, involved in carrying or transporting the subject the GSK-3β inhibitors.

In reference to the treatment of prostate cancer with GSK-3β inhibitors, a “therapeutically effective amount” refers to that amount which has the effect of (1) preventing the tumor from occurring or decreasing the risk of developing cancer, (2) reducing the size of the tumor, (3) inhibiting (that is, slowing to some extent, preferably stopping) tumor metastasis, (4) inhibiting to some extent (that is, slowing to some extent, preferably stopping) tumor growth, and (5) relieving to some extent (or, preferably, eliminating) one or more symptoms associated with the cancer (e.g., pain caused by bone metastasis), Prophylactic treatment is preferably administered to patients at risk for developing cancer. Risk factors include age (e.g. men above 60), race/ethnicity (African American and Latino descent), family history (especially if the patent has a father or brother with prostate cancer), genetic predisposition (e.g. BRCA1 or BRCA2 gene mutations), prior bowel cancer, and high insulin-like growth factor levels.

The amount that is therapeutically effective will depend upon the patient's size and gender, the condition to be treated, the severity of the condition and the result sought. The therapeutically effective dose can readily be determined using standard techniques known to those skilled in the art and the examples discussed herein.

As discussed more fully below, in the context of lithium in the treatment of cancer, preliminary results indicate that a therapeutically effective amount was found to include about 2 mg/kg of body weight in mice. It is estimated that this results in serum level of about 0.02 mEq/L in the animals (Moore 2002). Based on these preliminary results, therapeutically effective dosage levels include those substantially below those used for treatment of bipolar disorders with lithium in humans, which are reported to result in a serum level between about 0.2 and 1.5 mEq/L (Noble 2005), and is usually over 0.8 mEq/L.

In the present invention, a therapeutically effective dose of lithium is estimated to include those that result in serum levels of less than about 0.2 mEq/L. In one aspect, the therapeutically effective dose is less than about 0.1 mEq/L. In still another aspect, the therapeutically effective dose is less than about 0.05 mEq/L. And, in still a further aspect, the therapeutically effective dose is less than about 0.02 mEq/L.

In the context of prostate cancer treatment, it is contemplated that some of the GSK-3β inhibitors of the present invention may be used with other anti-neoplastic agents. As used herein, the phrase “anti-neoplastic agent” is synonymous with “chemotherapeutic agent” or “anti-cancer” and refers to compounds that prevent cancer cells from multiplying (i.e. anti-proliferative agents). Such compounds include, but are not limited to, paclitaxel, docetaxel, vinblastine, vincristine, vindesine, and vinorelbine; see for example the review: Cancer, Principles and Practice of Oncology, Lippincott-Raven Ed. (1997), 467-483. Platinum derivatives used in clinical practice include, but are not limited to cisplatin, carboplatin, oxaliplatin, nedaplatin, and lobaplatin; see review Cancer, Principles and Practice of Oncology, Lippincott-Raven Ed. (1997), 418-432. Other potential anti-neoplastic agents include alkylating agents (including, without limitation, nitrogen mustards, ethylenimine derivatives, alkyl sulfonates, nitrosoureas and triazines), uracil mustard, chlormethine, cyclophosphamide, ifosfamide, melphalan, chlorambucil, pipobroman, triethylene-melamine, triethylenethiophosphoramine, busulfan, carmustine, lomustine, streptozocin, dacarbazine, and temozolomide, antimetabolites (including, without limitation, folic acid antagonists, pyrimidine analogs, purine analogs and adenosine deaminase inhibitors), methotrexate, 5-fluorouracil, floxuridine, cytarabine, 6-mercaptopurine, 6-thioguanine, fludarabine phosphate, pentostatine, and gemcitabine, natural products and their derivatives (for example, vinca alkaloids, antitumor antibiotics, enzymes, lymphokines and epipodophyllotoxins), vinblastine, vincristine, vindesine, bleomycin, dactinomycin, daunorubicin, doxorubicin, epirubicin, idarubicin, ara-C, paclitaxel, mithramycin, deoxyco-formycin, mitomycin-C, L-asparaginase, interferons (IFN-alpha), etoposide, and teniposide. Other anti-proliferative cytotoxic agents are navelbene, CPT-11, anastrazole, letrazole, capecitabine, reloxafine, cyclophosphamide, ifosamide, and droloxafine.

Microtubule affecting agents interfere with cellular mitosis and are well known in the art for their anti-cancer activity. Microtubule affecting agents useful in the invention include, but are not limited to, allocolchicine (NSC 406042), Halichondrin B (NSC 609395), colchicine (NSC 757), colchicine derivatives (e.g., NSC 33410), dolastatin 10 (NSC 376128), maytansine (NSC 153858), rhizoxin (NSC 332598), paclitaxel (Taxol® NSC 125973), Taxol® derivatives (e.g., derivatives (e.g., NSC 608832), thiocolchicine NSC 361792), trityl cysteine (NSC 83265), vinblastine sulfate (NSC 49842), vincristine sulfate (NSC 67574), natural and synthetic epothilones including but not limited to epothilone A, epothilone B, and discodermolide (see Service, (1996) Science, 274:2009) estramustine, nocodazole, MAP4, and the like. Examples of such agents are also described in the scientific and patent literature, see, e.g., Bulinski (1997) J. Cell Sci. 110:3055 3064; Panda (1997) Proc. Natl. Acad. Sci. USA 94:10560-10564; Muhlradt (1997) Cancer Res. 57:3344-3346; Nicolaou (1997) Nature 387:268-272; Vasquez (1997) Mol. Biol. Cell. 8:973-985; Panda (1996) J. Biol. Chem. 271:29807-29812.

Additional anti-cancer agents include, melphalan, hexamethyl melamine, thiotepa, cytarabine, idatrexate, trimetrexate, dacarbazine, L-asparaginase, camptothecin, topotecan, bicalutamide, flutamide, leuprolide, pyridobenzoindole derivatives, interferons, and interleukins. Preferred classes of antiproliferative cytotoxic agents are the EGFR inhibitors, Her-2 inhibitors, CDK inhibitors, and Herceptin (trastuzumab). Some especially preferred anti-proliferative cytostatic agents are paclitaxel, cis-platin, carboplatin, epothilones, gemcytabine, CPT-11,5-fluorouracil, tegafur, leucovorin, and EGFR inhibitors such as Iressa®. (ZD 1839, 4-(3-chloro-4-fluorophenylamino)-7-methoxy-6-(3-(4-morpholinyl)propoxy)quinazoline and OSI-774 (4-(3-ethynylphenylamino)-6,7-bis(2-methoxyethoxy)quinazoline).

### Materials and Methods

1. Cell Culture, Antibodies, and Reagents

Androgen-responsive LNCaP cells, androgen-insensitive C4-2 cells, AR-null PC-3, and human embryonic kidney (“HEK”) 293 cell lines were described previously (Liao 2003, Liao 2004). These cell lines represent the clinical spectrum of prostate cancers. More specifically, LNCaP and C4-2 are AR-positive while PC-3 is AR null. LNCaP cells are androgen responsive but C4-2 and PC-3 cells are androgen-independent. All cell lines express a wild type Rb gene but have a distinct genetic background regarding p53. LNCaP and its derivative C4-2 cells harbor a wild type p53gene; in contrast, a p53 mutation was detected in PC-3 cell line (reviewed in Sobel 2005)

More specifically, the LNCaP, PC-3, and HEK293 cells were obtained from the Marican Type Culture Collection (Manassas, Va.). The androgen-insensitive cell line C4-2 was obtained from UroCor, Inc. (Oklahoma City, Okla.). The cells and were maintained in a humidified atmosphere of 5% CO₂, RPMI 1640 supplemented with 10% fetal bovine serum (“FBS”) with antibiotics (Invitrogen, Carlsbad, Calif.), except that the HEK293 cells were maintained in DMEM media.

The GSK-3β inhibitors SB216763 and SB415286, antibodies for cyclins, cdc6 and cdc25C, actin, and p21^{CIP1} were purchased from Santa Cruz Biotech (Santa Cruz, Calif.). Other antibodies were purchased from Cell Signaling (Beverly, Mass.). GSK-3β inhibitor BIO (6-bromoindirubin-3′-oxime (“BIO”)) was purchased from EMD Biosciences (San Diego, Calif.). Lithium chloride (“LiCl”), 0.4% trypan blue solution, sodium chloride (“NaCl”) and potassium chloride (“KCl”), as well as other chemicals were purchased from Sigma (St. Louis, Mo.). Where indicated, the inhibitor was added from a 1000-fold concentrated stock in the solvent. Control cultures received similar amounts of the solvent only. Final concentrations of the solvent did not exceed 0.1%.

2. Cell Counting, Clonogenic Assay and Cell Cycle Analysis

For cell counting assay, cells were seeded at the density of 5×10⁴ cells in 12-well plates in duplicates and allowed for attachment overnight. LiCl was added to the medium at various doses indicated in the figures. Cells were harvested daily for three days and the number of viable cells was counted using a hemocytometer after staining in trypan blue solution (Liao 2003).

For clonogenic assays, 1.0×10³ cells were seeded in a 35-mm dish and cultured in a medium containing LiCl at the indicated doses. The cultures were monitored daily for colony formation. On day 14, the cultures were washed with phosphate-buffered saline (“PBS”), fixed, and stained as described previously (Liao 2005). The colonies were counted and photographed under an inverted microscope.

For cell cycle analysis, serum-starved cells were left untreated or treated with different inhibitors for various time periods as indicated in the figures. After trypsinization, the cells were fixed and stained with propidium iodide (“PI”). Cell cycle distribution of PI-labeled cells was analyzed on a fluorescence activated cell sorter (Model EPICS XL-MCL, Beckman-Coulter Co., Fullerton, Calif.) as described previously (Miyake 2001).

3. MTT and BrdU Incorporation Assays

For MTT-based cell growth assays, cells seeded in 96-well plates were serum-starved for 48 hours, and were then treated with vehicle or different reagents for different time periods as indicated in the figures. MTT assay were carried out using a pre-assembled kit (Sigma) according to the manufacturer's manual.

For BrdU incorporation assays, cells were seeded and serum starved as discussed herein. After treatment with various reagents for different time periods as indicated in the figures, the cells were then incubated with BrdU for two hours. BrdU-labeled cells were detected using an ELISA-based colormeric kit (Roche, Indianapolis, Ind.) according to the procedure provided by the manufacturer.

4. Affymetrix cDNA-Based Microarray and RT-PCR

Androgen-insensitive C4-2 cells and HEK293 cells were used for cDNA-based microarray analysis. After serum starvation for 24 hours, cells were left untreated or treated with LiCl (10 mM) in serum free media for 30 minutes whereas the control cells received PBS in the same media. Then 10% FBS was brought back to both control and LiCl pre-treated cells and incubated for another 6 hours. At the end of treatment, total cellular RNAs were extracted using Trizol-based protocol as described (Liao 2005).

Genechip® arrays (Affymetrix Inc, Santa Clara, Calif.) used herein contain almost 45,000 probe sets representing more than 39,000 transcripts derived from approximately 33,000 well substantial human genes. The in vitro-transcription labeling is conducted using the EnzoBio array high yield RNA transcript labeling kit (Affymetrix). Ten micrograms of total RNAs were used for the first strand cDNA synthesis using the SuperscriptChoice system (Invitrogen).

Genechips were hybridized for 16 hours at 45° C. and 60 rpm in Genechip hybridization oven Model 640. After washing the Genechip, the signal intensity was detected using the Agilent Gene Array scanner.

RT-PCR was conducted to assess cell cycle-related gene expression at the mRNA level. Cells were serum-starved and then treated with the inhibitors as indicated for overnight. Total cellular RNAs were extracted and RT-PCR was carried out as described previously (Liao 2005). The primer sequences for cell cycle-related genes were listed in the table below and 28S ribozyme RNA was set as the internal control as described (Liao 2005).

TABLE 1
PCR primer sequences

| | |
|---|---|
| Gene | sequence (forward/backward) |

| | | |
|---|---|---|
| cyclin A | 5′-accccaagagtggagttgtg-3′ | (SEQ ID No.1) |
| | /5′-ggaaggcattttctgatcca-3′ | (SEQ ID No.2) |
| cyclin E | 5′-caggtttggagtgggacagt-3′ | (SEQ ID No.3) |
| | /5′-ctccattgcacactggtgac-3′ | (SEQ ID No.4) |
| cdc6 | 5′-tctgattcccaagagggttg-3′ | (SEQ ID No.5) |
| | /5′-ctgcctgatcaagagcatca-3′ | (SEQ ID No.6) |
| cdc25C | 5′gaacaggccaagactgaagc-3′ | (SEQ ID No.7) |
| | /5′-gcccctggttagaatcttcc-3′ | (SEQ ID No.8) |
| p21^{CIP1} | 5′-gacaccactggagggtgact-3′ | (SEQ ID No.9) |
| | /5′-caggtccacatggtcttcct-3′ | (SEQ ID No.10) |
| cdc2 | 5′-ttttcagagctttgggcact-3′ | (SEQ ID No.11) |
| | /5′-ccattttgccagaaattcgt-3′ | (SEQ ID No.12) |

The following PCR conditions were used: 95° C. for 45 seconds; 56° C. for 30 seconds; 72° C. for 60 seconds running in a total of 25 cycles. The PCR products were separated onto a 2% agarose gel and visualized with ethidium bromide under UV light (Liao 2005).

5. Western Blot Analysis

Cells seeded in 100 mm plates were serum-starved and then treated with GSK-3β inhibitors as indicated in the figures and described herein. Western blot was carried out as described previously (Liao 2004, Liao 2003, and Liao 2005). Briefly, cells were pellets and lysed in radio-immunoprecipitation assay (“RIPA”) buffer containing protease inhibitors (Half™ Protease Inhibitor Cocktail Kit, PIERCE, Rockford, Ill.). Proteins were separated on SDS-PAGE gels and transferred to Immuno-Blot™ PVDF membrane (BIO-RAD, Hercules, Calif.). Membranes were blocked in a tris-buffered solution plus Tween 20 (“TBST”) solution with 5% nonfat dry milk and incubated with primary antibodies overnight at 4° C. Immunoreactive signals were detected by horseradish peroxidase-conjugated secondary antibodies (Santa Cruz Biotech., Santa Cruz, Calif.) followed by SuperSingnal® WestFeml® Maximum Sensitivity Substrate (PIERCE).

6. Small Interference RNA Synthesis and Transfection

The small interference RNA against GSK-3β was synthesis using a transcription-based protocol (Ambion, Austin, Tex.). The sequences and procedures were described previously (Liao 2004, Liao 2003), of which the #4 siRNA duplex having SEQ ID NO. 13 (AAGAAUCGAGAGCUCCAGAUC) was chosen for silencing GSK-3β in this study. A negative siRNA duplex with scrambled sequences were purchased from Ambion and used as control. Transfection of the siRNA at a final concentration of 50 nM in the media was carried out with OligoFectamine™ agent (Invitrogen) as described (Liao 2004, Liao 2003, Liao 2005).

The gene-specific inhibition of gene expression by double-stranded ribonucleic acid is generally described in Fire et al., U.S. Pat. No. 6,506,559, which is incorporated by reference. Exemplary use of siRNA technology is further described in McSwiggen, Published U.S. Patent Application No. 2003/01090635; Reich et al., Published U.S. Patent Application No. 2004/0248174; Chi, Published U.S. Patent Application No. 2005/0026286; and Fosnaugh, Published U.S. Patent Application No. 2003/0148507, which are incorporated by reference.

7. Animal Experiments and Immunohistochemistry

All animal studies were conducted under an approved Institutional Animal Care and Use Committee protocol. Single cell suspensions (2×10⁶ cells in 0.1 ml of RPMI1640 medium plus 10% FBS) were inoculated subcutaneously (s.c.) into the rear flanks (two sites/mouse) of 6 to 7 weeks old male nude mice (Charles River, Wilmington, Mass.). For protocol A, mice were randomly divided into two groups (5 animals each). On next day after inoculation, one group of animals received i.p. injection of LiCl at a daily dose of 2 mg/kg body weight. LiCl was dissolved in PBS. The control groups of animals received PBS alone in the same volume. Xenograft development and growth were monitored for 8 weeks. The tumor dimensions were measured twice a week. The tumor volume was calculated by the formula of L×W×H×0.5236 (Miyake 2001). At the end of treatment, xenograft tumors were extirpated, and the final tumor size and weight, as well as animal weight were measured. For protocol B, treatment began until tumor became palpable or 50 mm³ in size, and LiCl or PBS was injected (5 animals each group) as above for a two-week course. Tumor size and weight were measured at the end of treatment. For BrdU in vivo labeling, the animals were injected i.p. with 0.5 ml of a 10-mM BrdU solution (Invitrogen) 2 hours before sacrifice. For experiments involving LNCaP-BclxL SB216763 subline cells (Liao 2005) were inoculated as above, and SB216763 treatment was started two weeks later when palpable xenografts were established. The SB216763 for animal use was purchased from Biomol (Plymouth Meeting, Pa.) and dissolved in a formula containing 50% N,N-dimethylacetamide (“DMA”), 45% polyethylene glycol 400 (“PEG-400”) and 5% Tween-80. SB216763 (2 mg/kilogram/bodyweight) or the solvent in 20 μl volume was injected i.p. three times a week. Tumor growth was monitored as discussed herein.

To detect BrdU labeled cells in xenografts, paraffin-embedded tumor sections were stained for incorporated BrdU using ZYMED® BrdU Staining Kit (Invitrogen). To detect apoptotic cell death in xenograft, terminal deoxynucleotidyl transferase dUTP Nick End Labeling (“TUNEL”) assay was conducted using Apo-BrdU-IHCTM In Situ DNA Fragmentation Assay Kit (BioVision, Mountain View, Calif.). Both assays were carried out according to the procedures described by the manufacturers.

7. Statistical Analysis

All cell culture-based experiments were repeated two or three times. Colony formation, cell cycle distribution, RT-PCR, Western blot and immunostaining results are presented from a representative experiment. The mean and standard deviation (“SD”) from two experiments for cell growth, BrdU labeling, S-phase cell population and tumor growth were shown. The significant differences between groups were analyzed as described previously (Liao, 2003, Liao 2004, Liao 2005) using the SPSS computer software (SPSS Inc., Chicago, Ill.).

### EXAMPLE 1

### GSK-3β Inhibition of Cell Growth In Vitro

This example investigated whether GSK-3β inhibitors could suppress prostate cancer cell growth using three common cell lines (AR-positive, androgen-responsive LNCaP cells, androgen insensitive C4-2 cells, and AR-null PC-3 cells). Cell growth was assessed using three different methods: trypan blue-based counting of viable cells, MTT, and colony formation. Four structurally-unrelated GSK-3β inhibitors, LiCl, SB216763, and SB415286, as well as indirubin analog BIO, were used to ensure the specificity of GSK-3β inhibition. The specificities for GSK-3β inhibition were established previously (Coehn 2004, Phiel 2001, Sato 2004). The inhibitors or vehicle were added once into the cell culture medium during the experiments.

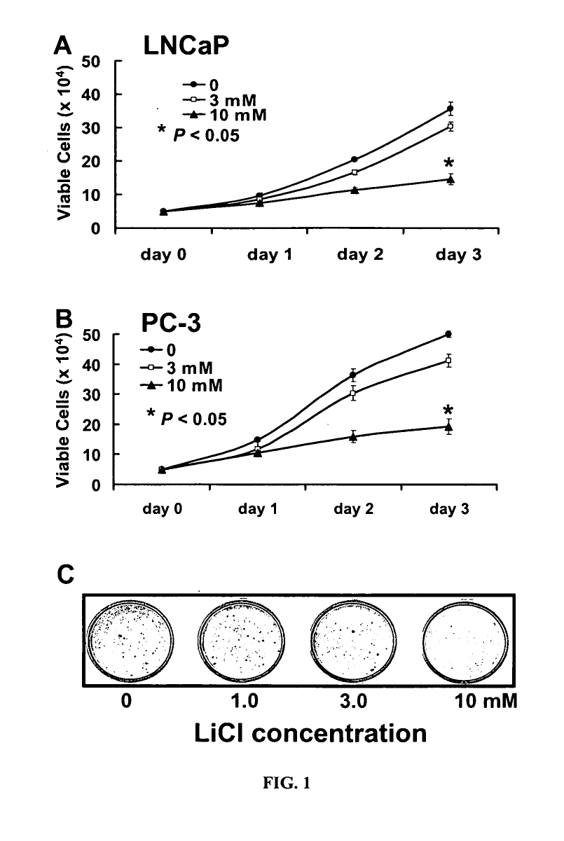

First, the effect of LiCl on cell growth was investigated. AR-positive, androgen-dependent LNCaP cells or AR-null PC-3 cells were seeded in a 12-well plate overnight and then treated with or without LiCl in different doses. As shown in FIGS. 1A and B, LiCl had no obvious inhibition at a dose of less than 3 mM for up to three days in both cell lines. However, LiCl significantly inhibited cell growth at 10 mM. At this concentration, the mean doubling-time for PC-3 cells increased significantly from 21.5 hours (no treatment) to 30.2 hours (treatment), while for LNCaP cells the mean doubling-time increased from 23.7 hours (no treatment) to 33.3 hours (treatment).

Next, the clonogenic assay was used to test the effect of LiCl on cell colony formation. The clonogenic survival fractions of the cells cultured in the presence or absence of increasing concentration of LiCl were measured in two cell lines, androgen refractory PC-3 (data shown) and androgen-responsive LNCaP (data not shown). As shown in FIG. 1C, LiCl at 10 mM significantly suppressed the surviving fractions, which was consistent with the data from cell counting assay (as seen in FIGS. 1A&B). Similar results were attained for the LNCaP cells. Taken together, these results indicate that LiCl suppresses cell growth in a dose-dependent and time-dependent manner.

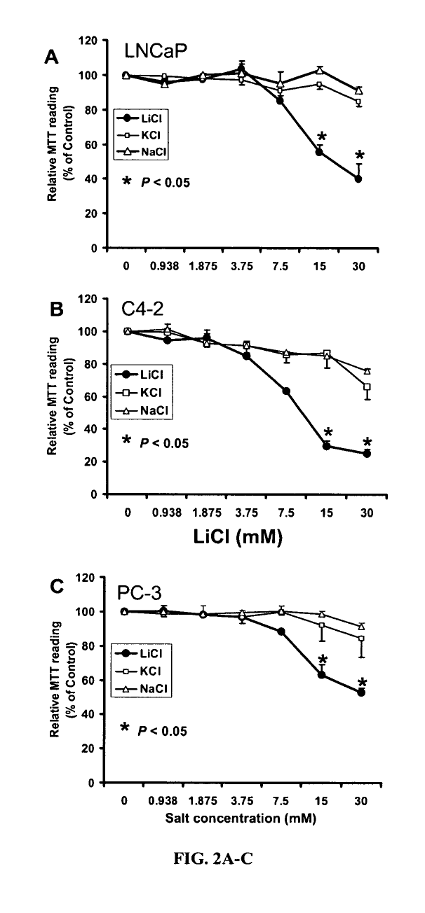

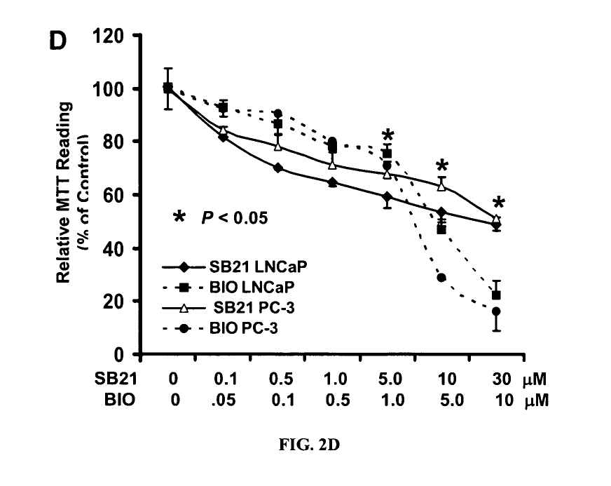

To rule out the eventual intervention of the chloride anion and the osmotic disturbance, the effect of LiCl was compared with sodium chloride (“NaCl”) and potassium chloride (“KCl”) at equimolecular concentrations in both LNCaP and PC-3 cell lines. In addition, the androgen-insensitive C4-2 cell line was included. To easily handle the experiments while multiple comparisons were taken in place, a MTT assay was used to assess cell growth. As shown in FIG. 2A-2C, NaCl or KCl did not cause any significant inhibition when used at the same concentration as LiCl, while LiCl induced a dramatic inhibition upon all three cell lines tested. These results were in agreement with the cell counting and clonogenic assays (FIG. 1).

Lastly, two GSK-3β-specific small chemical inhibitors, SB216763 and BIO, were used to verify that LiCl-induced suppression of cell growth is due to GSK-3β inhibition. LNCaP cells were seeded as above and then treated with increasing doses of the inhibitors in a full culture medium. Cell growth was assessed three days later by MTT assay. As shown in FIG. 2D, both SB216763 and BIO significantly suppressed cell growth in a dose-responsive manner. These results confirmed the fact that inhibition of GSK-3β activity suppresses cell growth in cancer in vitro.

### EXAMPLE 2

### GSK-3β Inhibitors Disrupt S-Phase Progression

Suppression of cell growth may result from cell cycle disturbance. Previous reports showed that GSK-3β inhibitors induce cell cycle arrest in some cell lines (Smits 1999, Damiens 2001). To determine if GSK-3β inhibitors interfere with cell cycle progression in prostate cancer cells, a cell cycle analysis in LNCaP, PC-3, and C4-2 cells after LiCl treatment was performed.

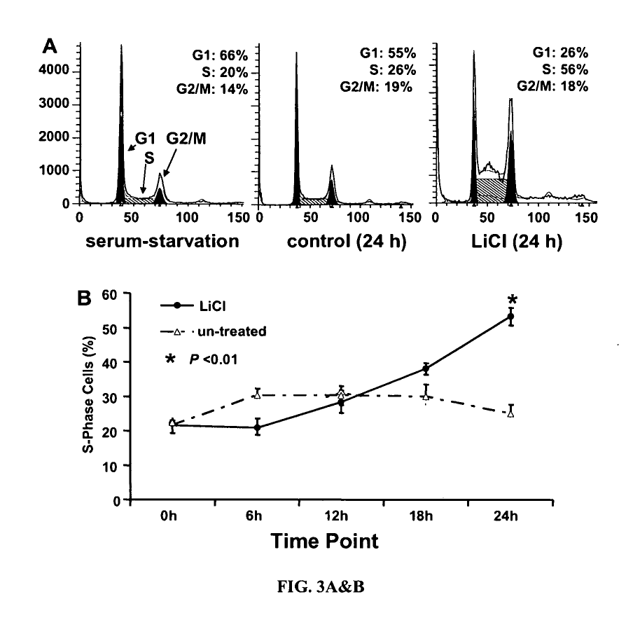

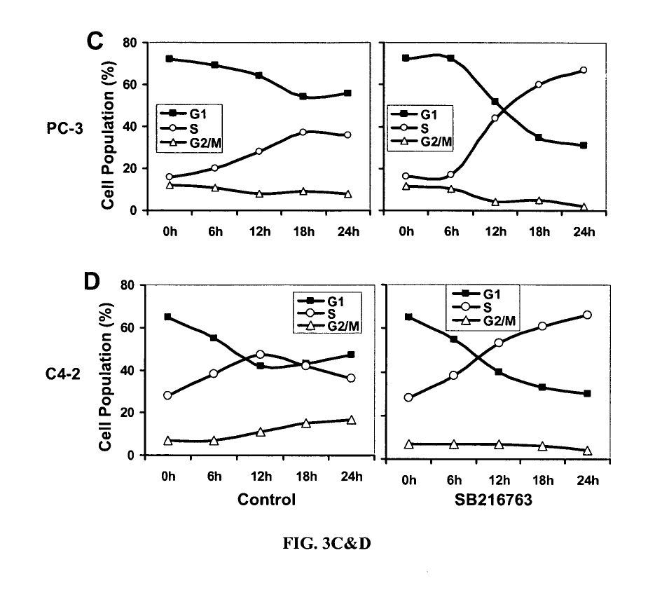

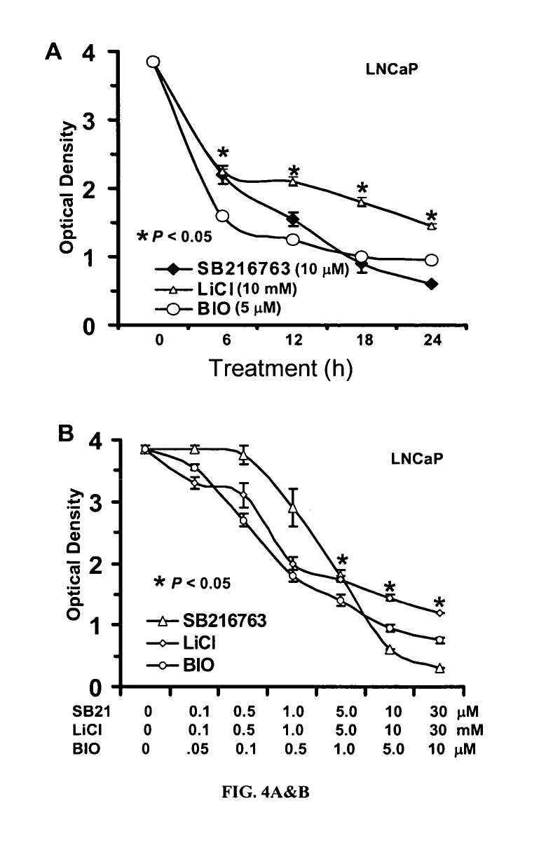

LNCaP cells were first serum-starved for 24 hours, and were then released by returning them to full culture media in the presence or absence of LiCl for up to 24 hours. Cell cycle distribution was analyzed by flow cytometry (“FCM”). As shown in FIG. 3A, after serum starvation most of the cells were arrested in G1-phase. When serum was added back in the culture (the control), cells re-entered into cell cycle. However, the portion of S-phase cells was dramatically increased in LiCl-treated population (FIG. 3A, right panel). As shown in FIG. 3B, when analyzing this S-phase accumulation in a 6-hour intervals, it was found that S-phase cells accumulated gradually in LiCl-treated group comparing to the control group.

The effect of GSK-3β SB216763 on cell cycle distribution in AR-null PC-3 and androgen-insensitive C4-2 cell lines were also examined. Similar to LiCl, SB216763 arrested these cells in S-phase after 12 hours treatment (FIGS. 3C and 3D). These data indicate that GSK-3β inhibitor-induced suppression of cell growth is due to a cell cycle arrest at S-phase.

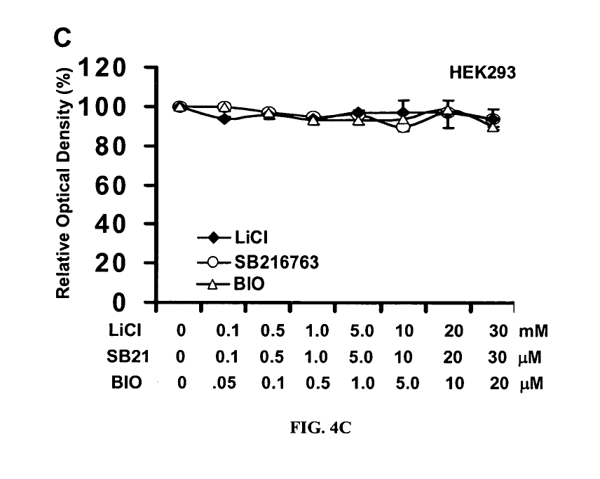

DNA replication or synthesis is the major event in S-phase of a cell cycle. Therefore, it is possible that GSK-3β inhibitors disrupt DNA synthesis that subsequently causes an S-phase arrest. To test this hypothesis, a BrdU incorporation assay was conducted to determine if cellular DNA synthesis was disrupted by GSK-3β inhibitors. BrdU-labeled cells were detected by an ELISA-based method. LNCaP cells were serum-starved as above and then treated with three different inhibitors for different time periods followed by a two-hour BrdU incubation to label newly-synthesized DNA. In a time-course experiment, GSK-3β inhibitors significantly reduced the number of BrdU-labeled cells after a six-hour treatment (FIG. 4A). Meanwhile, those inhibitors also showed a significant dose-responding effect on BrdU labeling (FIG. 4B).

To examine if GSK-3β inhibitors have a broad effect on DNA synthesis, HEK293 were included in this BrdU incorporation assay. In contrast to prostate cancer cells, HEK293 cells did not respond to GSK-3β inhibitor-induced decrease in BrdU labeling (FIG. 4C). These results indicate that GSK-3β inhibitors disrupt cellular DNA synthesis, which might be a cell-specific event.

### EXAMPLE 3

### GSK-3β Inhibitors Down-Regulate DNA Replication-Related Genes

For mammalian cells, DNA replication or synthesis occurs during S-phase. Successful completion of DNA replication and progression through S-phase requires coordinated action of numerous factors (reviewed in Takeda 2005). In brief, a multi-protein complex called pre-replication complex (“pre-RC”) forms at replication origins on chromatin DNA by origin recognition complex (“ORC”) and initiation factors cdc6 and Cdt1 before DNA replication process starts. During DNA replication, the pre-RC recruits a second set of replication factors, including minichromosome maintenance (“MCM”) proteins. Recent reports showed that cdc6 is critical in loading MCM proteins during DNA replication and has a dual role in G1-S and G2-M transitions (Takeda 2005, Cook 2002). Levels of cdc6 protein are selectively higher in proliferating but not quiescent mammalian cells and its expression is regulated via an E2 factor (“E2F”)-dependent transcriptional mechanism (Yan 1998). In addition, cyclin-dependent kinases (“CDK”) 2 and 4 together with their partners cyclin A and E are required for successful completion of S-phase (Coverley 2002, Blow 2005). Like cdc6, cyclin A and E are also E2F-responsive genes and their expression increases during G1/S transition (Schulze 1995, Ohtani 1995). CDK2 and GSK-3β phosphorylate and thus promote cyclin E degradation via the proteasome pathway (Welcker 2003). Deletion of cyclin E expression causes quiescent mouse embryonic fibroblast (“MEF”) cells to re-enter into S-phase, indicating that cyclin E is essential for DNA replication (Geng 2003). During S-phase progression, cyclin A/CDK2 complex is responsible for phosphorylation of the pre-RC components and is required for terminating DNA replication and entering mitosis with activation of cdc25C (Hutchins 2004, Mitra 2004). Disruption of cyclin A gene resulted in early embryonic death, demonstrating its fundamental role in cellular function (Murphy 1997).

Throughout the cell cycle, CDK activities are negatively controlled by CDK inhibitors. There are two families of CDK inhibitors. The first family includes p21^{CIP1}, p27^{KIP1} and p57^{KIP2}, and the second family includes p15, p16, p18 and p19, etc. (reviewed in Sherr 1999). They inhibit most of cyclin/CDK complexes, of which p21^{CIP1} is essential in DNA damage-induced growth arrest. Overexpression of p21^{CIP1} protein causes cell cycle arrest at S-phase and thus suppresses cell growth (Ogryzko 1997), although p21^{CIP1}-induced G1 or G2 arrest was reported (Niculescu 1998). As a common target for diverse signals that induce cell growth arrest and differentiation, p21^{CIP1} expression is regulated at both transcriptional and post-transcriptional mechanisms (Gartel 1999). GSK-3β phosphorylates and then promotes proteolysis of p21^{CIP1} protein (Rossig 2002).

This example investigated the genes that are altered by GSK-3β inhibition. This approach started with a genome-wide screening of gene expression with cDNA Microarray to identify the altered genes, which was verified by RT-PCR and Western blot at mRNA and protein levels, respectively. After sequestered in serum-free media for 24 hours, LNCaP cells were left untreated or treated with LiCl in a full culture media for 18 hours. The total cellular RNAs were extracted from the cells for cDNA Microarray analysis. Since GSK-3β inhibitors induced an S-phase arrest, genes that are related to S-phase progression were investigated.

In line with the cell cycle analysis as seen in FIG. 3, microarray analysis revealed a group of altered genes that are closely related to DNA replication after LiCl treatment of LNCaP cells as shown in the table below:

TABLE 1
cDNA Microarray for LiCl-induced changes

| | | | |
|---|---|---|---|
| | fold | treatment | control |
| Gene Probe | change | signal | signal |

Down-regulation

| | | | | |
|---|---|---|---|---|
| Cyclin A2 | 203418_at | −4.19 | 364.7 | 1529.8 |
| cdc6 | 203967_at | −3.47 | 327 | 1136 |
| cdc6 | 203968_s_at | −3 | 377.9 | 1132.5 |
| Cyclin E2 | 205034_at | −2.99 | 423.8 | 1268.1 |
| Cyclin E2 | 211814_s_at | −2.18 | 391.2 | 852.7 |
| cdc25c | 217010_s_at | −5.99 | 34.7 | 207.8 |
| cdc25c | 205167_s_at | −2.74 | 161.2 | 442.3 |
| cyclin B2 | 202705_at | −1.4 | 1693.9 | 2379.1 |
| p27KIP1 | 209112_at | −1.17 | 1171.7 | 1365.8 |

Up-regulation

| | | | | |
|---|---|---|---|---|
| p21CIP1 | 202284_s_at | 5.7 | 2457.2 | 430.9 |
| cyclin D1 | 203665_at | 2.07 | 609.3 | 294.7 |
| cyclin D1 | 208712_at | 1.84 | 1568.2 | 853.9 |
| cyclin G2 | 202770_s_at | 1.87 | 606.9 | 325.3 |
| p57KIP2 | 213348_at | 1.79 | 246.4 | 137.6 |

The most dramatic changes were down-regulation of cdc6, cdc25C, cyclins A and E, as well as up-regulation of CDK inhibitor p21^{CIP1}. As mentioned earlier, cyclin A and E together with cdc6 are the most critical players for DNA replication in S-phase (Takeda 2005) and cdc25C phosphatase is a gate opener for entering mitosis (Hutchins 2004). No significant changes were found for other cell cycle related proteins, such as cyclin B, cdc2, p27^{KIP1} and p57^{KIP2}, although a slight increase of cyclin D1 was observed. These alterations of cell cycle genes were not observed in HEK293 cells after LiCl treatment (microarray data not shown).

Based on the list of altered genes identified by microarray analysis, a series of experiments using RT-PCR technique were conducted to verify the alterations induced by LiCl treatment. As before, LNCaP cells were serum-starved for 24 hours and then treated with the inhibitors. In addition to LiCl and SB216763 as used earlier, SB415286 was included in these verification experiments.

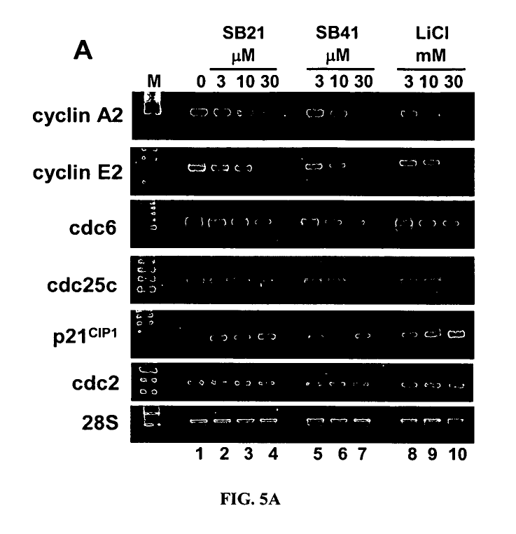

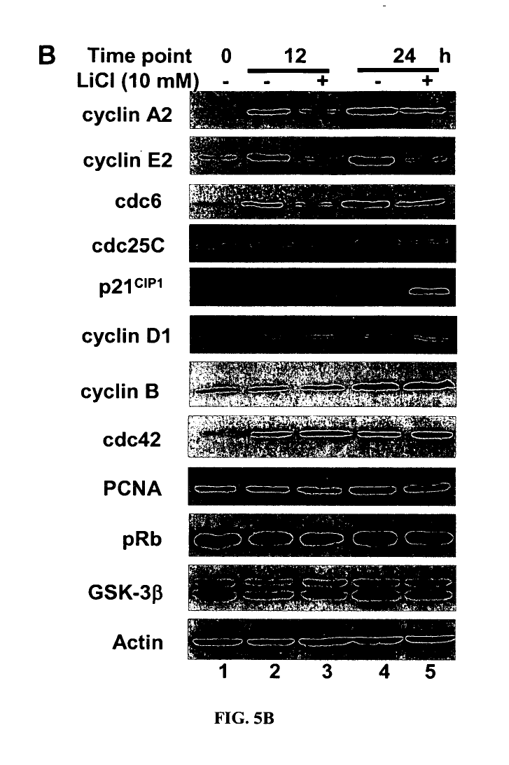

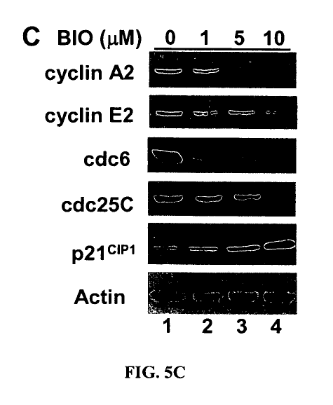

As shown in FIG. 5A, all three inhibitors suppressed the expression of those target genes (cyclin A and E, as well as cdc6 and cdc25C). In parallel, GSK-3β inhibitor treatment increased p21^{CIP1} expression. These changes are in a dose-dependent fashion and were consistent with the cDNA microarray data.

To verify those alterations at a protein level, two series experiments were conducted using Western blot technique. The first involved a time-course experiment. After serum starvation, LNCaP cells were left untreated or treated as before with LiCl for up to 24 hours. Cells were harvested at 18-24 hours. As shown in FIG. 5B, most of the target genes were expressed at a relatively low level after serum starvation (at 0 time point) but increased significantly after serum addition. However, LiCl treatment dramatically suppressed serum-induce increase of those genes (cyclins A and E, cdc6 and cdc25C). In addition, p21^{CIP1} expression was increased significantly in LiCl-treated cells compared to the untreated control. For other cell cycle-related genes, such as cyclin B, PCNA, pRb, cdc2 and cdc42, as well as GSK-3β, no significant change was observed after LiCl treatment. Consistent with the cDNA microarray data, the protein levels of cyclin D1 was slightly higher in LiCl-treated cells compared to the control.

Next, a dose-response experiment was performed. Following serum starvation for 24 hours, LNCaP cells were treated with BIO at different doses for another 24 hours. Control cells received the vehicle as treatment. As shown in FIG. 5C, BIO treatment significantly reduced the protein levels of those genes in a dose-dependent fashion, except that p21^{CIP1} expression was increased dramatically. These results were consistent to LiCl-induced effect in the time-course experiments.

Lastly, GSK-3β siRNAs were used to determine if GSK-3β gene silencing leads to cell growth suppression and alterations of those aforementioned genes as induced by GSK-3β inhibitors in androgen-independent cells. The siRNA duplex against GSK-3β gene was described above and (Liao 2004, Liao 2003). AR-null PC-3 cells were transfected with the GSK-3β siRNA or a negative siRNA. Cell growth was monitored for up to three days after transfection. Cells were harvested at the end of experiments for evaluating protein levels of target genes by Western blot.

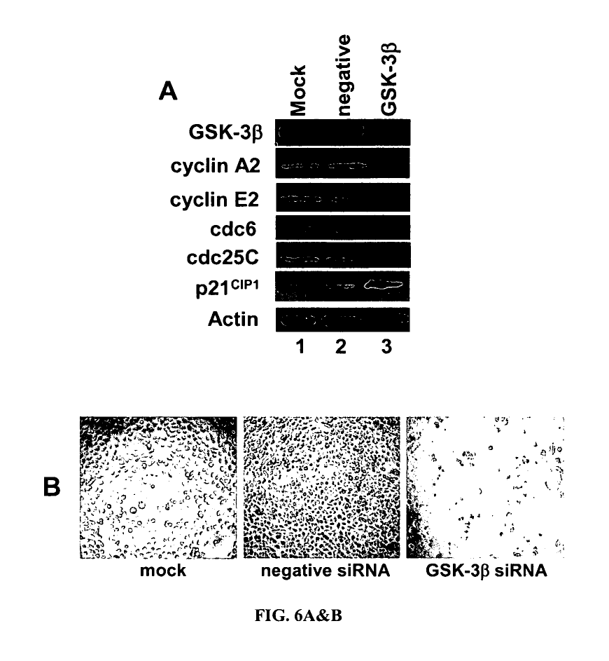

As shown in top panel of FIG. 6A, GSK-3β siRNA eliminated GSK-3β expression while the negative siRNA had no effect. These data was in agreement with previous publications (Liao 2004, Liao 2003). As expected, cell growth was significantly suppressed by the GSK-3β siRNA but not the negative siRNA compared to the mock transfection (FIG. 6B). In parallel to siRNA-induced elimination of GSK-3β protein expression, protein levels of those aforementioned genes, such as cdc6, cyclin A and E, as well as cdc25C, decreased significantly in GSK-3β siRNA-transfected AR-null PC-3 cells compared to the controls. Consistently, p21 CPI protein level increased largely after GSK-3β silencing.

All together, these data demonstrated that inhibition of GSK-3β activity suppresses cell growth by disrupting DNA synthesis and S-phase progression, which is due to reduced expression of DNA replication-related genes and increased expression of p21^{CIP1}. This occurs in both androgen dependent prostate cancers and in androgen independent cancers.

### EXAMPLE 4

### GSK-3β Inhibitors Suppress Tumor Growth In Vivo

This example investigated GSK-3β inhibitor-induced suppression of cancer cell growth in vivo. A mouse xenograft model was used to examine the effect of GSK-3β inhibitor on tumor growth in two different strategies (protocol A and B). In both protocols, LiCl was delivered at a daily dose i.p. of 2 mg/kg of body weight. This dose level is more than 100-fold lower than a therapeutic dose for mental diseases in mouse (Noble 2005) or human (Moore 2002). In both protocols, no obvious abnormality of daily activities or any sign of side effect was observed from LiCl-treated animals. There was no significant difference of animal body weight and daily activities between treatment and control groups.

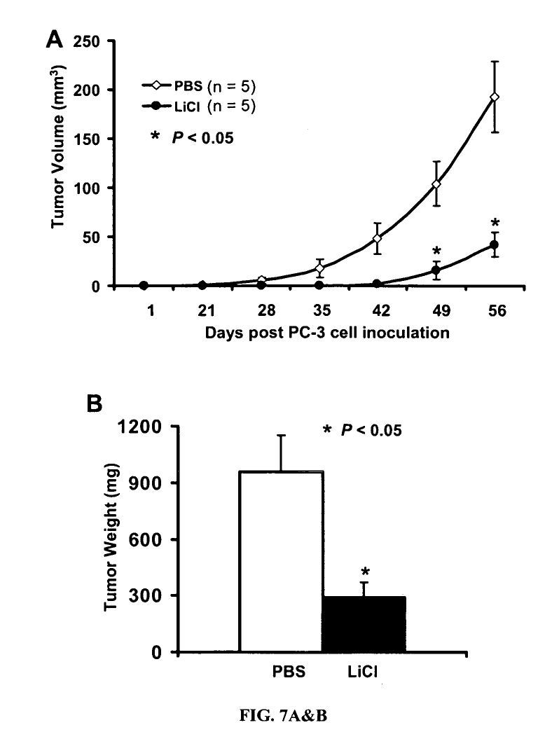

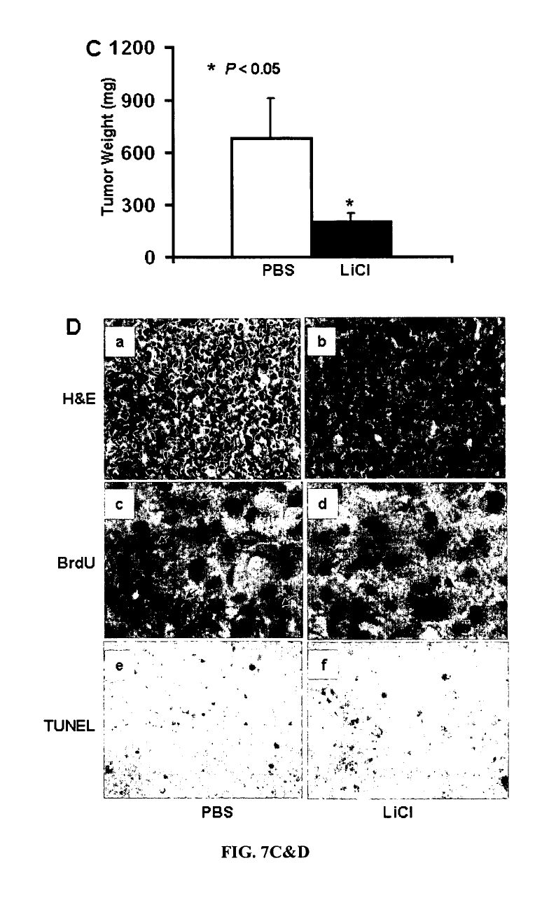

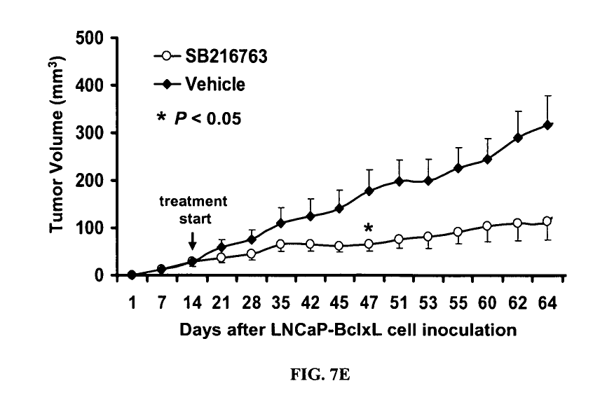

In protocol A, LiCl treatment was started on the same day when AR-null PC-3 cancer cells were inoculated into animals. AR-null PC-3 xenografts were developed around 4 weeks after inoculation in PBS-treated control animals. However, LiCl treatment significantly delayed xenograft development until 6-7 weeks after inoculation (FIG. 7A). Comparing the tumor volume and weight at the end of 8-week treatment between the two groups, LiCl treatment resulted in a significant reduction compared to PBS control (FIGS. 7A and 7B).

In protocol B, animals were treated with LiCl at 2 mg/kg for a two-week course when PC-3 xenografts were palpable or about 30 mm³ in size. Tumor weight was measured at the endpoint of treatment and was compared between LiCl-treated and PBS control groups. As shown in FIG. 7C, LiCl treatment also significantly reduced tumor weight compared to PBS control.

Next, the DNA synthesis associated with LiCl-induced tumor suppression in vivo was also investigated. DNA synthesis was measured by an in vivo BrdU incorporation assay. Meanwhile, apoptotic events, if any, were detected using TUNEL assay. BrdU was injected two hours before animal was sacrificed. Tumor samples were processed for anti-BrdU immunostaining. As shown in FIG. 7D, LiCl treatment significantly reduced the BrdU labeling compared to PBS control in xenografts (panel C vs. D). However, TUNEL staining did not show any difference between the two groups (panel E and F).

Finally, the suppression of xenograft tumor growth derived from an LNCaP subline that over expresses a potent anti-apoptotic Bcl-xL gene (as it was often seen in advanced prostate cancer patients) was tested with a GSK-3β inhibitor (Krajewska 1996). This LNCaP subline was recently generated and termed as LNCaP-BclxL (Liao 2005). Overexpression of Bcl-xL gene greatly enhanced xenograft development. As shown in FIG. 7E, LNCaP-BclxL xenografts became palpable within two weeks after inoculated in castrated nude mice. When the tumor was palpable, animals were treated i.p. with SB216763 at a dose of 2 mg/kg bodyweight every two days. Control animals received the vehicle only. In control group, LNCaP-BclxL xenografts displayed a faster growing feature. However, SB216763 treatment largely suppressed tumor growth compared to the control animals. The difference in tumor volume between SB216763-treated and vehicle control animals became significant after a 5-week treatment. No side effects were observed in SB216763-treated or vehicle control animals.

Taken together, this data demonstrated for the first time that inhibition of GSK-3β activity suppresses prostate cancer tumor growth in vivo. The GSK-3β inhibitors were surprisingly effective in androgen-refractory prostate cancers. Further, the mechanism of action relate to the inhibition of DNA synthesis in S-phase.

The following references are incorporated by reference to the extent that they are needed to teach one skilled in the art how to make and use the invention described herein.
Jope R S, Johnson G V. The glamour and gloom of glycogen synthase kinase-3. Trends Biochem Sci 2004; 29:95-102
Kim L, Kimmel A R. GSK3, a master switch regulating cell-fate specification and tumorigenesis. Curr Opin Genet Dev 2000; 10(5):508-14.
Ross S E, Erickson R L, Hemati N, MacDougald O A. Glycogen synthase kinase 3 is an insulin-regulated C/EBPalpha kinase. Mol Cell Biol 1999; 19(12):8433-41.
Yang S D. Proline-directed protein kinase FA as a potential target for diagnosis and therapy of human cancers. Curr Cancer Drug Targets 2004; 4(7):591-6.
Manoukian A S, Woodgett J R. Role of glycogen synthase kinase-3 in cancer: regulation by Wnts and other signaling pathways. Adv Cancer Res 2002; 84:203-29.
Liao X, Thrasher J B, Holzbeierlein J, Stanley S, Li B. Glycogen synthase kinase-3beta activity is required for androgen-stimulated gene expression in prostate cancer. Endocrinology 2004; 145:2941-9.
Mazor M, Kawano Y, Zhu H, Waxman J, Kypta R M. Inhibition of glycogen synthase kinase-3 represses androgen receptor activity and prostate cancer cell growth. Oncogene 2004; 23:7882-92.
Wang L, Lin H K, Hu Y C, Xie S, Yang L, Chang C. Suppression of androgen receptormediated transactivation and cell growth by the glycogen synthase kinase 3 beta in prostate cells. J Biol Chem 2004; 279:32444-52.
Salas T R, Kim J, Vakar-Lopez F, et al. Glycogen synthase kinase-3 beta is involved in the phosphorylation and suppression of androgen receptor activity. J Biol Chem 2004; 279:19191-200. Sun et al. GSK-3β inhibitors suppress tumor growth
Liao X, Zhang L, Thrasher J B, Du J, Li B. Glycogen synthase kinase-3beta suppression eliminates tumor necrosis factor-related apoptosis-inducing ligand resistance in prostate cancer. Mol Cancer Ther 2003; 2:1215-22.
Hoeflich K P, Luo J, Rubie E A, Tsao M S, Jin O, Woodgett J R. Requirement for glycogen synthase kinase-3beta in cell survival and NF-kappaB activation. Nature 2000; 406:86-90.
Takeda D Y, Dutta A. DNA replication and progression through S phase. Oncogene 2005; 24:2827-43.
Cook J G, Park C H, Burke T W, et al. Analysis of Cdc6 function in the assembly of mammalian prereplication complexes. Proc Natl Acad Sci USA 2002; 99:1347-52.
Yan Z, DeGregori J, Shohet R, et al. Cdc6 is regulated by E2F and is essential for DNA replication in mammalian cells. Proc Natl Acad Sci USA 1998; 95:3603-8.
Coverley D, Laman H, Laskey R A. Distinct roles for cyclins E and A during DNA replication complex assembly and activation. Nat Cell Biol 2002; 4:523-8.
Blow J J, Dutta A. Preventing re-replication of chromosomal DNA. Nat Rev Mol Cell Biol 2005; 6:476-86.
Schulze A, Zerfass K, Spitkovsky D, et al. Cell cycle regulation of the cyclin A gene promoter is mediated by a variant E2F site. Proc Natl Acad Sci USA 1995; 92:11264-8.
Ohtani K, DeGregori J. Nevins J R. Regulation of the cyclin E gene by transcription factor E2F1. Proc Natl Acad Sci USA 1995; 92:12146-50.
Welcker M, Singer J, Loeb K R, et al. Multisite phosphorylation by Cdk2 and GSK3 controls cyclin E degradation. Mol Cell 2003; 12:381-92.
Geng Y, Yu Q, Sicinska E, et al. Cyclin E ablation in the mouse. Cell 2003; 114:431-43. Sun et al. GSK-3β inhibitors suppress tumor growth.
Hutchins J R, Clarke P R. Many fingers on the mitotic trigger: post-translational regulation of the Cdc25C phosphatase. Cell Cycle 2004; 3:41-5.
Mitra J, Enders G H. Cyclin A/Cdk2 complexes regulate activation of Cdk1 and Cdc25 phosphatases in human cells. Oncogene 2004; 23:3361-7.
Murphy M, Stinnakre M G, Senamaud-Beaufort C, et al. Delayed early embryonic lethality following disruption of the murine cyclin A2 gene. Nat Genet 1997; 15:83-6.
Sherr C J, Roberts J M. CDK inhibitors: positive and negative regulators of G1-phase progression. Genes Dev 1999; 13:1501-12.
Ogryzko V V, Wong P, Howard B H. WAF1 retards S-phase progression primarily by inhibition of cyclin-dependent kinases. Mol Cell Biol 1997; 17:4877-82.
Niculescu A B 3rd, Chen X, Smeets M, Hengst L, Prives C, Reed S I. Effects of p21(CIP1/Waf1) at both the G1/S and the G2/M cell cycle transitions: pRb is a critical determinant in blocking DNA replication and in preventing endoreduplication. Mol Cell Biol 1998; 18:629-43.
Gartel A L, Tyner A L. Transcriptional regulation of the p21(WAF1/CIP1) gene. Exp Cell Res 1999; 246:280-9.
Rossig L, Badorff C, Holzmann Y, Zeiher A M, Dimmeler S. Glycogen synthase kinase-3 couples AKT-dependent signaling to the regulation of p21CIP1 degradation. J Biol Chem 002; 277:9684-9.
Cohen P, Goedert M. GSK3 inhibitors: development and therapeutic potential. Nat Rev Drug Discov 2004; 3:479-87.
Phiel C J, Klein P S. Molecular targets of lithium action. Annu Rev Pharmacol Toxicol 2001; 41:789-813. Sun et al. GSK-3β inhibitors suppress tumor growth
Huot J, Nosal G, Radoucu-Thomas S. Effects of lithium chloride on normal and neoplastic cells in vitro. Experientia 1972; 28:456-7.
Cohen Y, Chemit A, Cohen Y, Sirota P, Modan B. Cancer morbidity in psychiatric patients: influence of lithium carbonate treatment. Med Oncol 1998; 15:32-6.
Erdal E, Ozturk N, Cagatay T, Eksioglu-Demiralp E, Ozturk M. Lithium-medicated downregulation of PKB/Akt and cyclin E with growth inhibition in hepatocellular carcinoma cells. Int J Cancer 2005; 115:903-10.
Liao X, Tang S, Thrasher J B, Griebling T L, Li B. Small-interfering RNA-induced androgen receptor silencing leads to apoptotic cell death in prostate cancer. Mol Cancer Ther 2005; 4-505-15.
Miyake H, Nelson C, Rennie P S and Gleave M E. Overexpression of insulin-like growth factor binding protein-5 helps accelerate progression to androgen-independence in the human prostate LNCaP tumor model through activation of phosphatidylinositol 3′-kinase pathway. Endocrinology 20001; 41:2257-65.
Sobel R E, Sadar M D. Cell lines used in prostate cancer research: a compendium of old and new lines—part 1. J Urol 2005; 173:342-59.
Sato N, Meijer L, Skaltsounis L, Greengard P, Brivanlou A H. Maintenance of pluripotency in human and mouse embryonic stem cells through activation of Wnt signaling by a pharmacological GSK-3-specific inhibitor. Nat Med 2004; 10:55-63.
Lochhead P A, Coghlan M, Rice S Q, Sutherland C. Inhibition of GSK-3 selectively reduces glucose-6-phosphatase and phosphatase and phosphoenolypyruvate carboxykinase gene expression. Diabetes 2001; 50:937-46. Sun et al. GSK-3β inhibitors suppress tumor growth
Smits V A, Essers M A, Loomans D S, Klompmaker R, Rijksen G, Medema R H. Inhibition of cell proliferation by lithium is associated with interference in cdc2 activation. FEBS Lett 1999; 457:23-7.
Damiens E, Baratte B, Marie D, Eisenbrand G, Meijer L. Anti-mitotic properties of indirubin-3′-monoxime, a CDK/GSK-3 inhibitor: induction of endoreplication following prophase arrest. Oncogene 2001; 20:3786-97.
Ghosh P K, Biswas N M, Ghosh D. Effect of lithium chloride on spermatogenesis and testicular steroidogenesis in mature albino rats: duration dependent response. Life Sci 1991; 48:649-57.
Noble W, Planel E, Zehr C, et al. Inhibition of glycogen synthase kinase-3 by lithium correlates with reduced tauopathy and degeneration in vivo. Proc Natl Acad Sci USA 2005; 102:6990-5.
Moore C M, Demopulos C M, Henry M E, et al. Brain-to-serum lithium ratio and age: an in vivo magnetic resonance spectroscopy study. Am J Psychiatry 2002; 159:1240-2.
Krajewska M, Krajewski S, Epstein J I, et al. Immunohistochemical analysis of bcl-2, bax, bcl-X, and mcl-1 expression in prostate cancers. Am J Pathol 1996; 148:1567-76.
Ryves W J, Harwood A J. The interaction of glycogen synthase kinase-3 (GSK-3) with the cell cycle. Prog Cell Cycle Res 2003; 5:489-95.
Diehl J A, Cheng M, Roussel M F, Sherr C J. Glycogen synthase kinase-3beta regulates cyclin D1 proteolysis and subcellular localization. Genes Dev 1998; 12:3499-511.
Yuan H, Mao J, Li L, Wu D. Suppression of glycogen synthase kinase activity is not sufficient for leukemia enhancer factor-1 activation. J Biol Chem 1999; 274:30419-23. Sun et al. GSK-3β inhibitors suppress tumor growth.
Ougolkov A V, Fernandez-Zapico M E, Savoy D N, Urrutia R A, Billadeau D D. Glycogen synthase kinase-3beta participates in nuclear factor kappaB-mediated gene transcription and cell survival in pancreatic cancer cells. Cancer Res 2005; 65:2076-81.
Bracken A P, Ciro M, Cocito A, Helin K. E2F target genes: unraveling the biology. Trends Biochem Sci 2004; 29:409-17.
Dimova D K, Dyson N J. The E2F transcriptional network: old acquaintances with new faces. Oncogene 2005; 24:2810-26.
Litovchick L, Chestukhin A, DeCaprio J A. Glycogen synthase kinase 3 phosphorylates RBL2/p130 during quiescence. Mol Cell Biol 2004; 24:8970-80.
Nerlov C. C/EBPalpha mutations in acute myeloid leukaemias. Nat Rev Cancer 2004; 4:394-400.
Ross S E, Erickson R L, Hemati N, MacDougald O A. Glycogen synthase kinase 3 is an insulin-regulated C/EBPalpha kinase. Mol Cell Biol 1999; 19:8433-41.
Tang Q Q, Gronborg M, Huang H, et al. Sequential phosphorylation of CCAAT enhancer binding protein beta by MAPK and glycogen synthase kinase 3beta is required for adipogenesis. Proc Natl Acad Sci USA 2005; 102:9766-71.
Timchenko N A, Wilde M, Nakanishi M, Smith J R, Darlington G J. CCAAT/enhancer binding protein alpha (“C/EBP” alpha) inhibits cell proliferation through the p21 (WAF-1/CIP-1/SDI-1) protein. Genes Dev 1996; 10:804-15.
Turowski P, Franckhauser C, Morris M C, Vaglio P, Fernandez A, Lamb N J. Functional cdc25C dual-specificity phosphatase is required for S-phase entry in human cells. Mol Biol Cell 2003; 14:2984-98.
Ozen M, Ittmann M. Increased expression and activity of CDC25C phosphatase and an alternatively spliced variant in prostate cancer. Clin Cancer Res 2005; 11:4701-6. Sun et al. GSK-3β inhibitors suppress tumor growth.
Feng D, Tu Z, Wu W, Liang C. Inhibiting the expression of DNA replication-initiation proteins induces apoptosis in human cancer cells. Cancer Res 2003; 63:7356-64.
Bai V U, Cifuentes E, Menon M, Barrack E R, Reddy G P. Androgen receptor regulates Cdc6 in synchronized LNCaP cells progressing from G1 to S phase. J Cell Physiol 2005; 204:381-7.
Yamamoto A, Hashimoto Y, Kohri K, et al. Cyclin E as a coactivator of the androgen receptor. J Cell Biol 2000; 150:873-80.

From the foregoing, it will be seen that this invention is one well adapted to attain all ends and objectives herein above set forth, together with the other advantages that are obvious and which are inherent to the invention. Further, since many possible embodiments may be made of the invention without departing from the scope thereof, it is to be understood that all matters herein set forth or shown in the drawings are to be interpreted as illustrative, and not in a limiting sense. While specific embodiments or examples have been shown and discussed, various modifications may of course be made, and the invention is not limited to such embodiments and examples except insofar as such limitations are included in the following claims. Further, it will be understood that certain features and subcombinations are of utility and may be employed without reference to other features and subcombinations. This is contemplated by and is within the scope of the claims.

What is claimed and desired to be secured by Letters Patent is as follows:

1. A method for treating an androgen-refractory prostate cancer in vivo in a subject in need comprising administering a therapeutically effective amount of a pharmaceutically acceptable glycogen synthase kinase-3β (“”) inhibitor to said subject.

2. The method of claim 1 wherein said GSK-3β inhibitor is a lithium salt is selected from the group consisting of lithium succinate, lithium citrate, lithium acetylsalicylate, lithium chloride, lithium carbonate, and lithium orotate.

3. The method of claim 1 wherein said GSK-3β inhibitor is selected from the group consisting of a lithium salt; GF109203X (2-[1-(3-Dimethylaminopropyl)-1H-indol-3-yl]-3-(1H-indol-3-yl)maleimide); RO318220 (2-[1-(3-(Amidinothio)propyl)-1H-indol-3-yl]-3-(1-methylindol-3-yl)maleimide•methanesulfonate); SB216763 (3-(2,4-Dichlorophenyl)-4-(1-methyl-1H-indol-3yl)-1H-pyrrole-2,5-dione); SB415286 (3-[(3-Chloro-4-hydroxyphenyl)amino]-4-(2-nitrophenyl)-1H-pyrrole-2,5-dione); 4-Benzyl-2-methyl-1,2,4-thiadiazolidine-3,5-dione (“TDZD-8”); 2-Thio(3-iodobenzyl-5-(1-pyridyl)-[1,3,4]oxadiazole (“TIBPO”); 2,4-Dibenzyl-5-oxothiadiazolidine-3-thione (“OTDZT”); and 4-(2-Amino-4-oxo-2-imidazolin-5-ylidene)-2-bromo-4,5,6,7-tetrahydropyrrolo[2,3-c]azepin-8-one (“10Z-Hymenialdisine”).

4. The method of claim 1 wherein said GSK-3β inhibitor comprises siRNA for GSK-3β.

5. The method of claim 1 wherein said therapeutically effective amount of GSK-3β inhibitor comprises about 2 mg/kg of body weight of a pharmaceutically acceptable lithium salt.

6. The method of claim 1 wherein said prostate cancer is inhibited by S-phase arrest after administration of said GSK-3β inhibitor.

7. The method of claim 1 wherein said subject comprises a human.

8. The method of claim 1 wherein said GSK-3β is also administered an anti-cancer agent or treated with radiotherapy.

9. The method of claim 8 wherein said anti-cancer agent is a microtubule affecting agent.

10. The method of claim 1 wherein said prostate cancer has metastasized to said patient bone, and wherein said treatment method further inhibits pain associated with said metastasized prostate cancer in said bone.

11. The method of claim 1 wherein said therapeutically effective amount of GSK-3β inhibitor is lithium, and said amount results in a lithium serum level of less than about 0.2 mEq/L

12. The method of claim 1 wherein said therapeutically effective amount of GSK-3β inhibitor is lithium, and said amount results in a lithium serum level of less than about 0.1 mEq/L.

13. The method of claim 1 wherein said therapeutically effective amount of GSK-3β inhibitor is lithium, and said amount results in a lithium serum level of less than about 0.02 mEq/L

14. The method of claim 1 wherein said administration step is performed prophylactically by administering said GSK-3β inhibitor to a subject at risk of developing prostate cancer.
